(19) United States
(12) Patent Application Publication
(10) Pub. No.: US 2014/0038204 A1
(43) Pub. Date: Feb. 6, 2014

(54) SELECTIVE MEASUREMENT OF ACTIVE HUMAN PROTEASE COAGULATION FACTORS

(71) Applicants: Baxter Healthcare S.A., Glattpark (Opfikon) (CH); Baxter International Inc., Deerfield, IL (US)

(72) Inventors: Alfred Weber, Vienna (AT); Andrea Engelmaier, Vienna (AT); Hans-Peter Schwarz, Vienna (AT)

(21) Appl. No.: 13/943,392

(22) Filed: Jul. 16, 2013

Related U.S. Application Data
(60) Provisional application No. 61/677,981, filed on Jul. 31, 2012.

(51) Int. Cl. G01N 33/86

(52) U.S. Cl. CPC G01N 33/86
USPC 435/7.4

(57) ABSTRACT

The present invention relates to a method for the selective determination of the concentration of an active human protease coagulation factor in a sample, comprising the steps of binding a specific inhibitor of the human protease coagulation factor to a solid phase, letting the human protease coagulation factor contained in the sample bind to the solid phase-bound inhibitor, and detecting the human protease coagulation factor bound to the solid phase-bound inhibitor with a detection reagent.

### CROSS-REFERENCE TO RELATED APPLICATIONS

This application claims priority benefit of U.S. provisional application No. 61/677,981, filed Jul. 31, 2012, which is herein incorporated by reference for all purposes.

### FIELD OF THE INVENTION

The present invention relates to a method for the selective determination of the concentration of an active human protease coagulation factor in a sample, comprising the steps of binding a specific inhibitor of the human protease coagulation factor to a solid phase, letting the human protease coagulation factor contained in the sample bind to the solid phase-bound inhibitor, and detecting the human protease coagulation factor bound to the solid phase-bound inhibitor with a detection reagent.

### BACKGROUND OF THE INVENTION

The advent of low molecular weight peptide substrates, so-called chromogenic substrates, opened a new era for the measurement of protease activity. Before this pioneering introduction of synthetic substrates for the measurement of enzymes, proteases were measured by using their naturally occurring high molecular weight substrates, mainly proteins. Although such a procedure mimicked the natural action of the protease as close as possible, such procedures still had certain limitations, especially as far as sensitivity and specificity were concerned. The use of chromogenic substrates clearly improved the sensitivity of the measurements, but could not fully resolve issues related to the selectivity of the chromogenic substrates. Thus, depending on the selectivity of a given chromogenic substrate, instead of selectively measuring the amidolytic activity of a defined protease, the sum of the activity of all proteases recognizing the substrate is obtained. This is especially true for e.g. the proteases of the intrinsic pathway of the human coagulation system, namely kallikrein, activated factor XII (FXIIa) and activated factor XI (FXIa). To increase the moderate selectivity of the chromogenic substrate PyrGlu-Pro-Arg-p-nitroanilide (S-2366) for plasma FXIa, a complex amidolytic procedure has been described, in which the application of specific inhibitors for kallikrein and FXIIa creates the selectivity required to measure FXIa in the presence of the two other proteases of the intrinsic pathway of the coagulation system.

Apart from the sometimes only moderate selectivity, the use of chromogenic substrates can be associated with two further obstacles: First, the activity measured with a chromogenic substrate represents a so-called amidolytic activity. This term relates to the fact that only a small peptide is used and split by the protease instead of the natural, in most cases high molecular weight substrate. Thus, the interaction of the protease with its natural substrate could not only be limited to the site where cleaving takes actually place, but also includes interaction sites that are located in some distance to the cleavage site. This will determine the selectivity of the protease's action on the protein but cannot be fully recovered by the small synthetic substrate. The occurrence of an event like those described above will result in a low agreement between the activities determined with a natural substrate and the activities measured with the chromogenic substrate. Human thrombin is a well-known example for such an event: Ongoing auto-activation leads to the loss of clotting activity, measured with its natural substrate fibrinogen, whereas the amidolytic activity remains nearly unaffected. Another obstacle possibly affecting the measurement of proteases with chromogenic substrates, especially when done in plasma-like samples and in particular in the presence of α2-macroglobulin is also linked to their small size: Chromogenic substrates will measure also α2-macroglobulin-bound proteases although the complexed proteases show no activity against high molecular weight substrates, since such substrates cannot enter the cage and come into contact with the inhibitor. This interference can be handled by e.g. destroying the inhibitory activity of α2-macroglobulin by incubation with methylamine, but this has to be considered whenever amidolytic activity of proteases has to be measured.

Therefore, a strong need exists to provide a novel assay for the determination of the concentration of active human protease coagulation factors in a sample. This assay should display improved selectivity and sensitivity.

This need is satisfied by providing the embodiments characterized in the claims.

### SUMMARY OF THE INVENTION

The present invention describes the use of solid phase-bound specific inhibitors of human protease coagulation factors for determining the concentration of active human protease coagulation factors in a sample. After incubation of the sample containing the respective human protease coagulation factor to be measured with the solid phase-bound inhibitor, the amounts of inhibitor-protease complex formed on the solid support are then directly measured by using specific detection reagents. The high selectivity of this assay format results (i) from the specificity of the inhibitor used for the inhibitor-protease complex formation, which is not changed after binding the protease to a solid support, and (ii) on the specificity of the detection reagent used to measure the amount of complex formed on the solid phase.

In particular, the present invention relates to a method for the selective determination of the concentration of an active human protease coagulation factor in a sample, comprising the steps of binding a specific inhibitor of the human protease coagulation factor to a solid phase; incubating the solid phase-bound inhibitor with the sample; incubating the human protease coagulation factor bound to the solid phase-bound inhibitor with a detection reagent binding to the human protease coagulation factor; determining the amount of detection reagent bound to the human protease coagulation factor bound to the solid phase-bound inhibitor; and determining the amount of active human protease coagulation factor in the sample from the amount of detection reagent determined in step (d).

### DETAILED DESCRIPTION OF THE INVENTION

In one aspect, the present invention relates to a method for the selective determination of the concentration of an active human protease coagulation factor in a sample, comprising the steps of:
(a) binding a specific inhibitor of the human protease coagulation factor to a solid phase;
(b) incubating the solid phase-bound inhibitor with the sample;
(c) incubating the human protease coagulation factor bound to the solid phase-bound inhibitor with a detection reagent binding to the human protease coagulation factor;
(d) determining the amount of detection reagent bound to the human protease coagulation factor bound to the solid phase-bound inhibitor; and
(e) determining the amount of active human protease coagulation factor in the sample from the amount of detection reagent determined in step (d).

In this context, the term “human protease coagulation factor” as used herein refers to components of the human blood coagulation pathway, having a protease activity. The human protease coagulation factor may be of natural or non-natural, e.g. recombinant origin. Further, the human protease coagulation factor may have one or more amino acid substitutions, additions or deletions as compared to the wild-type amino acid sequence, provided it retains its native function. Finally, the human protease coagulation factor may have one or more natural, e.g. posttranslational, or non-natural, e.g. chemical, modifications such as e.g. phosphorylations and/or sulfatations and/or PEGylation. In preferred embodiments of the method of the present invention, the human protease coagulation factor is selected from the group consisting of factor XIIa (FXIIa), factor XIa (FXIa), kallikrein, factor IIa (FIIa; prothrombin), factor Vila (FVIIa), and factor Xa (FXa), wherein FXa, FXIIa and FXIa are particularly preferred.

Further, the term “active human protease coagulation factor” as used herein refers to a human protease coagulation factor that is capable of exerting its native function, i.e., to specifically cleave its native substrate.

Specific inhibitors of human protease coagulation factors are not particularly limited, provided they display a high degree of specificity for the human protease coagulation factor to be measured. Respective inhibitors are known in the art. In preferred embodiments, the human protease coagulation factor and the corresponding specific protease inhibitor are selected from the group consisting of FXa and tissue factor pathway inhibitor (TFPI), FXIIa and corn trypsin inhibitor (CTI), FXIa and C1-inhibitor, kallikrein and C1-inhibitor, FIIa and heparin cofactor II, and FVIIa and antithrombin.

Means for binding a specific inhibitor of a human protease coagulation factor to a solid phase are known in the art and are not particularly limited. They include for example the incubation of the solid support with the inhibitor in a suitable buffer, e.g. phosphate-buffered saline (PBS), for an appropriate length of time, e.g. overnight, at an appropriate temperature, e.g. +4° C. Further suitable buffers and incubation parameters are known in the art.

The solid phase on which the method of the present invention is performed is not particularly limited. Moreover, suitable solid phases are known in the art. In a preferred embodiment, the solid phase is selected from the group consisting of microplates, gels, and microparticles, wherein microplates are particularly preferred, e.g. microplates having an absorptive surface such as, e.g., a NUNC™ Maxisorp plates.

In the context of the present invention, the specific inhibitor can be attached to the solid phase by adsorption, where it is retained by hydrophobic forces. Alternatively, the surface of the solid phase can be activated by chemical processes that cause covalent linkage of the inhibitor to the support.

If the solid phase is silicon or glass, the surface must generally be activated prior to attaching the inhibitor. Activated silane compounds such as triethoxy amino propyl silane, triethoxy vinyl silane, and (3-mercapto-propyl)-trimethoxy silane can be used to introduce reactive groups such as amino, vinyl, and thiol groups, respectively. Such activated surfaces can be used to link the inhibitor directly (in the cases of amino or thiol), or the activated surface can be further reacted with linkers such as glutaraldehyde, bis(succinimidyl)suberate, SPPD (succinimidyl 3-[2-pyridyldithio]propionate), SMCC (succinimidyl-4-[N-maleimidomethyl]cyclohexane-1-carboxylate), SLAB (succinimidyl [4-iodoacetyl]aminobenzoate), and SMPB (succinimidyl 4-[1-maleimidophenyl]butyrate) to separate the inhibitor from the surface. Vinyl groups can be oxidized to provide a means for covalent attachment. Vinyl groups can also be used as an anchor for the polymerization of various polymers such as poly-acrylic acid, which can provide multiple attachment points for the inhibitor. Amino groups can be reacted with oxidized dextrans of various molecular weights to provide hydrophilic linkers of different size and capacity. Examples of oxidizable dextrans include Dextran T-40 (molecular weight 40,000 Daltons), Dextran T-110 (molecular weight 110,000 Daltons), Dextran T-500 (molecular weight 500,000 Daltons), Dextran T-2M (molecular weight 2,000,000 Daltons), or Ficoll (molecular weight 70,000 Daltons). Additionally, polyelectrolyte interactions can be used to immobilize the inhibitor on the solid phase.

The solid phase can be any suitable material with sufficient surface affinity to bind the inhibitor. Useful solid supports include: natural polymeric carbohydrates and their synthetically modified, crosslinked, or substituted derivatives, such as agar, agarose, cross-linked alginic acid, substituted and cross-linked guar gums, cellulose esters, especially with nitric acid and carboxylic acids, mixed cellulose esters, and cellulose ethers; natural polymers containing nitrogen, such as proteins and derivatives, including cross-linked or modified gelatins; natural hydrocarbon polymers, such as latex and rubber; synthetic polymers, such as vinyl polymers, including polyethylene, polypropylene, polystyrene, polyvinylchloride, polyvinylacetate and its partially hydrolyzed derivatives, polyacrylamides, polymethacrylates, copolymers and terpolymers of the above polycondensates, such as polyesters, polyamides, and other polymers, such as polyurethanes or polyepoxides; inorganic materials such as sulfates or carbonates of alkaline earth metals and magnesium, including barium sulfate, calcium sulfate, calcium carbonate, silicates of alkali and alkaline earth metals, aluminum and magnesium; and aluminum or silicon oxides or hydrates, such as clays, alumina, talc, kaolin, zeolite, silica gel, or glass (these materials can be used as filters with the above polymeric materials); and mixtures or copolymers of the above classes, such as graft copolymers obtained by initializing polymerization of synthetic polymers on a pre-existing natural polymer. Nitrocellulose and nylon can also be used. All of these materials can be used in suitable shapes, such as films, sheets, tubes, particulates, or plates, or they can be coated onto, bonded, or laminated to appropriate inert carriers, such as paper, glass, plastic films, fabrics, or the like.

Alternatively, the solid phase can constitute microparticles. Appropriate microparticles can be selected by one skilled in the art from any suitable type of particulate material and include those composed of polystyrene, polymethylacrylate, polypropylene, latex, polytetrafluoroethylene, polyacrylonitrile, polycarbonate, or similar materials. Further, the microparticles can be magnetic or paramagnetic microparticles, so as to facilitate manipulation of the microparticle within a magnetic field.

Microparticles can be suspended in the mixture of reagents and sample or can be retained and immobilized by a support material. In the latter case, the microparticles on or in the support material are not capable of substantial movement to positions elsewhere within the support material. Alternatively, the microparticles can be separated from suspension in the mixture of reagents and sample by sedimentation or centrifugation. When the microparticles are magnetic or paramagnetic the microparticles can be separated from suspension in the mixture of reagents and sample by a magnetic field.

Incubation of the solid phase with the sample according to the present invention can be performed at suitable temperatures for a suitable length of time, e.g. for 60 minutes at room temperature. Further suitable time/temperature combinations are known to a person skilled in the art.

In a preferred embodiment, the solid phase is blocked prior to incubating the solid phase with the sample in step (b), in order to decrease unspecific adsorption of the human protease coagulation factor or other sample components to the solid phase. Suitable blocking agents are known in the art and are not particularly limited. They include for example bovine serum albumin (BSA), methylated BSA, human serum albumin, casein, hydrolyzed casein, nonfat dry milk, gelatin or milk powder in suitable buffers. A suitable blocking buffer is for example PBS containing 0.05% polysorbate 20 and 10 mg/mL BSA. Blocking can be performed at suitable temperatures for a suitable length of time, e.g. for 60 minutes at room temperature. Further suitable time/temperature combinations are known to a person skilled in the art.

In another preferred embodiment, the sample is diluted prior to incubation with the solid phase in step (b). Suitable dilution ratios depend on the expected concentration of the human protease coagulation factor in the sample and are known to a person skilled in the art. Further, suitable dilution buffers are known in the art and are not particularly limited. They include for example PBS. Preferably, the dilution buffer contains an inert protein to avoid absorptive losses of the analyte during the dilution procedure.

Suitable detection reagents that can be used in the method of the present invention are known in the art and are not particularly limited. They include for example compounds, compositions or molecules capable of specifically or substantially specifically (i.e. with limited cross-reactivity) binding to an epitope of the respective human protease coagulation factor. These agents (or ligands) are typically antibodies, such as monoclonal antibodies, or derivatives or analogs thereof, but also include, without limitation: Fv fragments; single chain Fv (scFv) fragments; Fab′ fragments; F(ab′)₂ fragments; humanized antibodies and antibody fragments; camelized antibodies and antibody fragments; chimeric antibodies; and multivalent versions of the foregoing. Multivalent capture agents also can be used, as appropriate, including, without limitation: monospecific or bispecific antibodies; such as disulfide stabilized Fv fragments. These agents also include, without limitation, aptamers, synthetic peptides, binding molecules, nucleic acids, etc. and are as known in the art. Suitable labels for the detection reagents to be used in the method of the present invention are not particularly limited and are known in the art. They include for example enzymatic labels such as peroxidase, alkaline phosphatase, glucose oxidase, or β-galactosidase; biotin; fluorescent molecules; and radiolabels. In a preferred embodiment, the detection reagent is an antibody specific for the respective human protease coagulation factor. Said antibody is preferably conjugated to peroxidase.

Incubation of the solid phase with a detection reagent can be performed at suitable temperatures for a suitable length of time, e.g. for 60 minutes at room temperature. Further suitable time/temperature combinations are known to a person skilled in the art. Further, suitable dilution ratios and buffers for diluting the detection reagent prior to incubation are known in the art and can be easily determined.

In a preferred embodiment, the solid phase is washed prior to and/or after incubating the solid phase with the detection reagent in step (c). Suitable washing buffers are known in the art and are not particularly limited. They include for example PBS containing 0.05% polysorbate 20.

Means for determining the amount of detection reagent bound to the solid phase are known in the art and are not particularly limited. In the case of peroxidase-labeled antibody conjugates, they include for example the incubation of the solid phase with a suitable chromogenic peroxidase substrate, e.g. the ready-to-use trimethylbenzidine peroxidase reagent SureBlue (KPL), or with a fluorogenic substrate, at a suitable temperature, e.g. at room temperature, for a sufficient amount of time to allow color formation. Subsequently, color or fluorochrome formation can be stopped, e.g. by addition of 1.5 M sulfuric acid, and the amount of formed color or fluorochrome determined, e.g. in an ELISA reader at 450 nm. Further suitable chromogenic or fluorogenic substrates, incubation times and temperatures, and means for measuring the amount of formed color are known to a person skilled in the art. Amplification systems commonly used in the art, including the biotin-avidin system, can also be employed in the context of the present invention.

Finally, means for determining the amount of active human protease coagulation factor in the sample are known in the art and are not particularly limited. They include for example the correlation of sample data to a suitable calibration curve that is e.g. established using known concentrations of human protease coagulation factor.

The figures show:

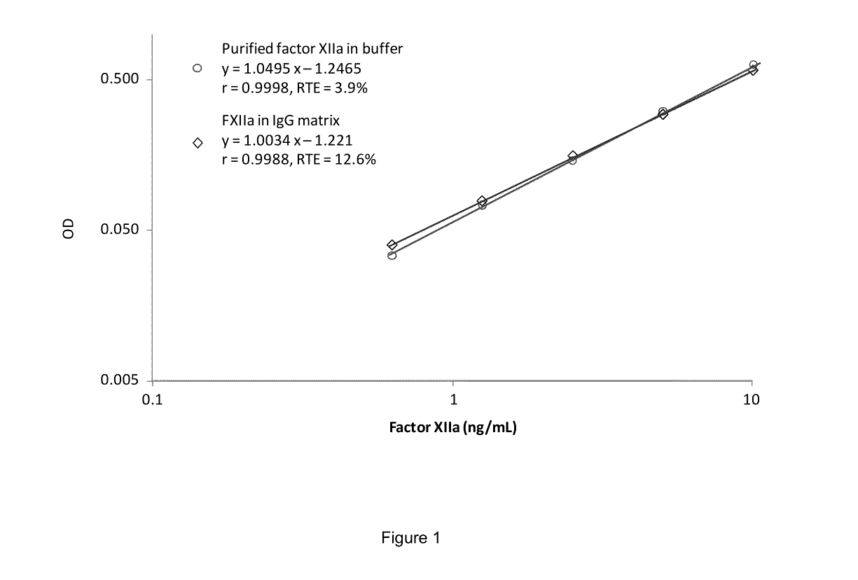

FIG. 1 shows a 5-point calibration curve for the measurement of human FXIIa in buffer and in the presence of 2.5 mg/mL human IgG.

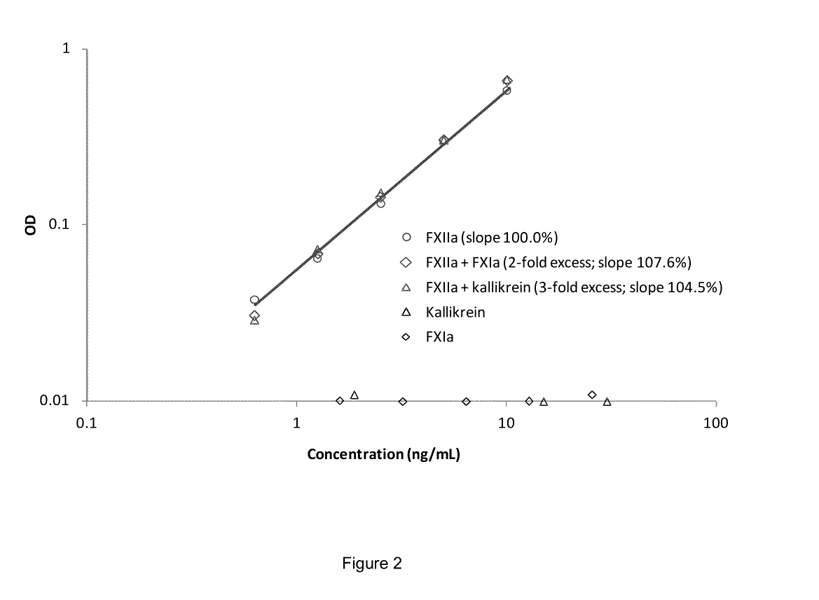

FIG. 2 shows dose-response curves for samples containing FXIIa alone, FXIIa and FXIa, FXIIa and kallikrein, kallikrein alone, and FXIa alone.

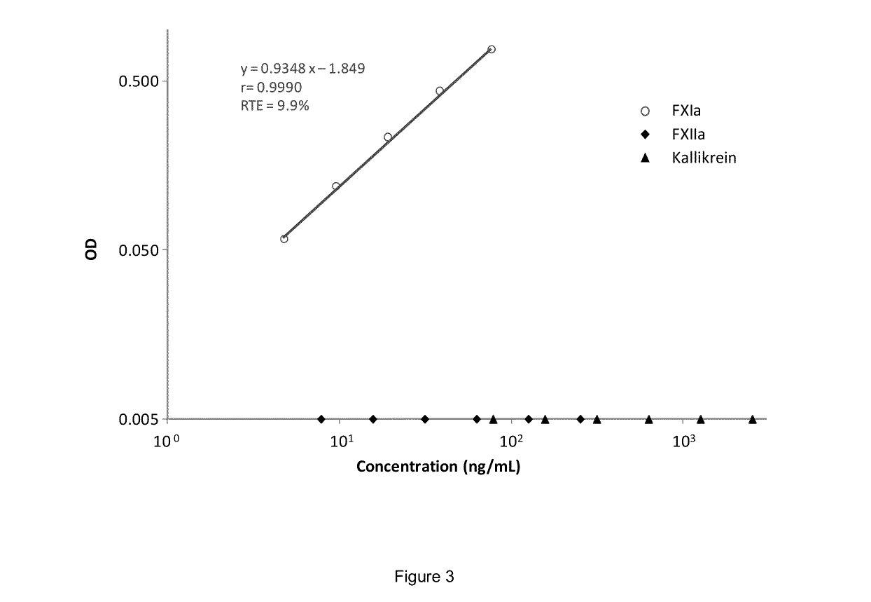

FIG. 3 shows a 5-point calibration curve obtained for a dilution series of FXIa and the response measured for the purified proteases kallikrein and FXIIa.

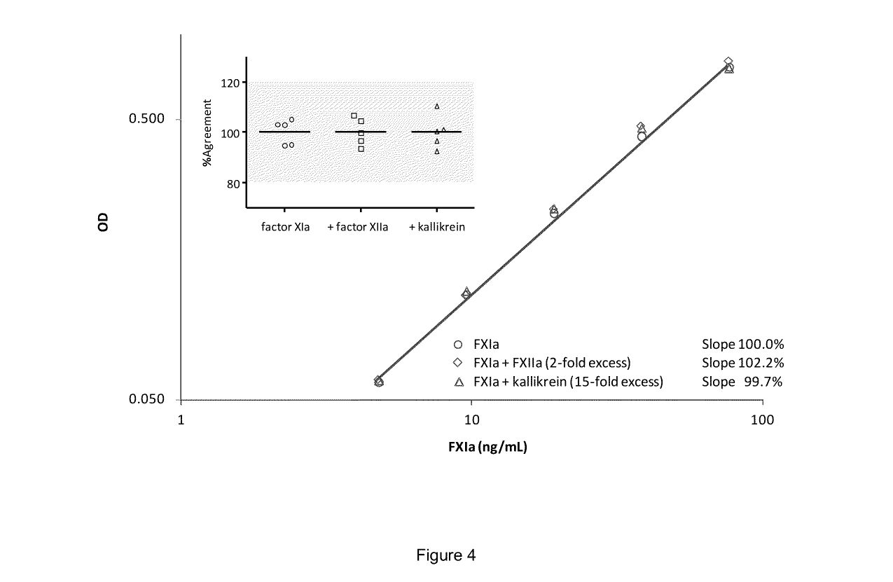

FIG. 4 shows dilution-response curves for FXIa and mixtures of FXIa with FXIIa and kallikrein.

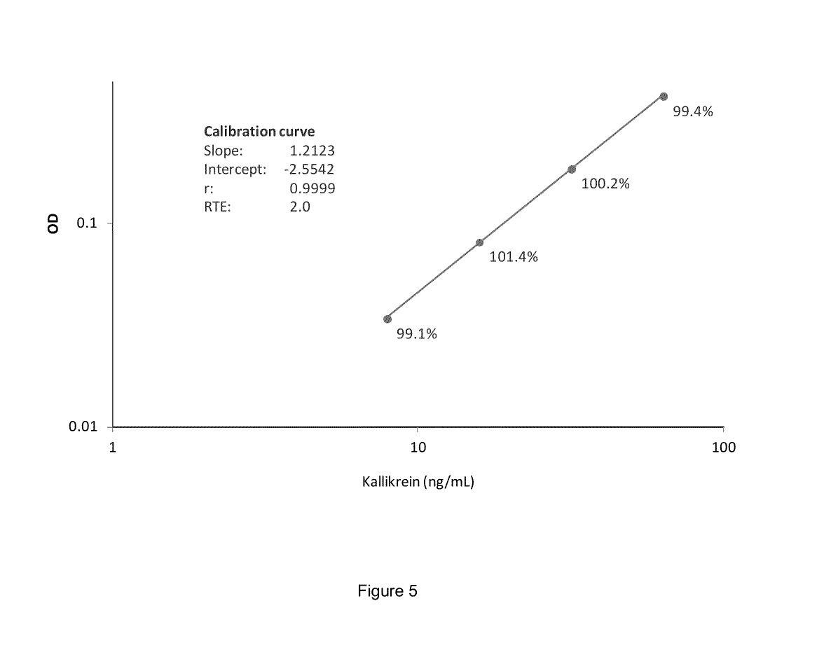

FIG. 5 shows a calibration curve for the measurement of kallikrein with C1-inhibitor.

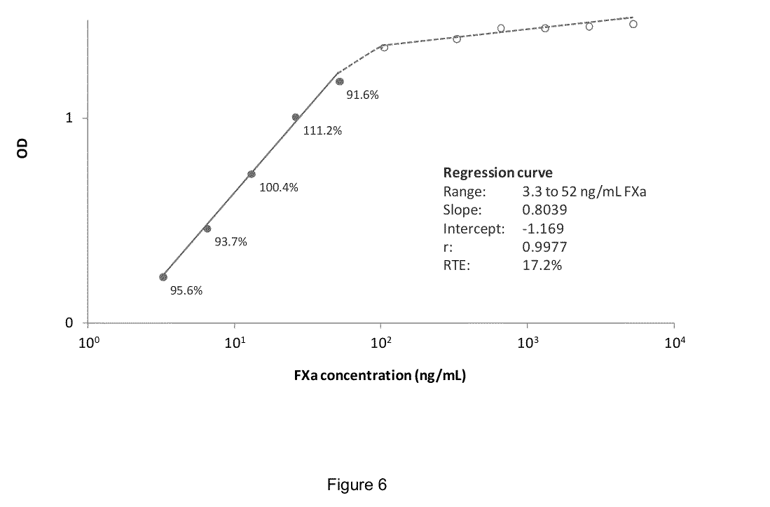

FIG. 6 shows the dose-response relation for a dilution series of FXa using plate-bound TFPI.

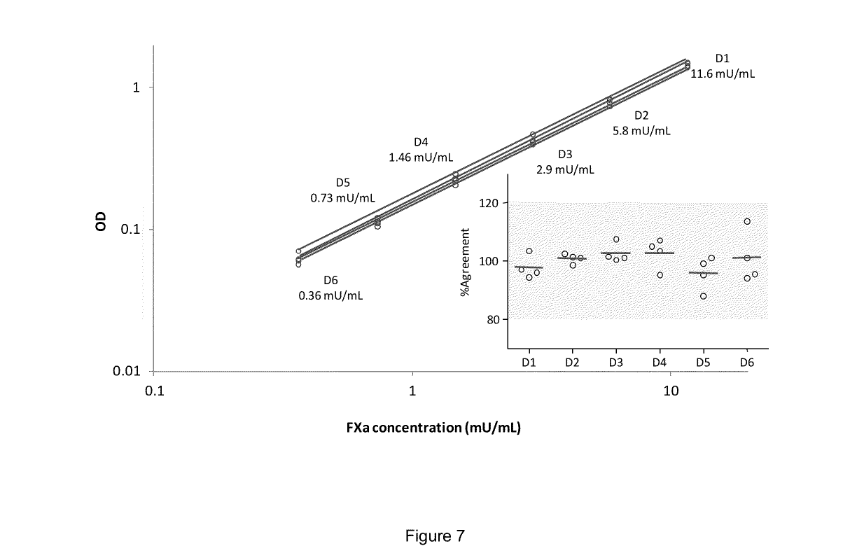

FIG. 7 shows four representative calibration curves for the measurement of FXa using plate-bound TFPI.

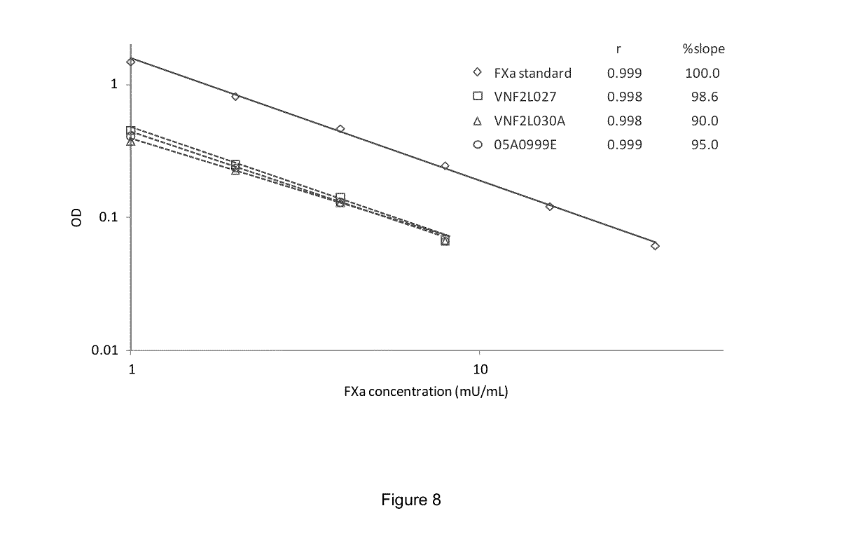

FIG. 8 shows dose-response curves for the FXa measurement with plate-bound TFPI done in three lots of an activated prothrombin complex concentrate (APCC).

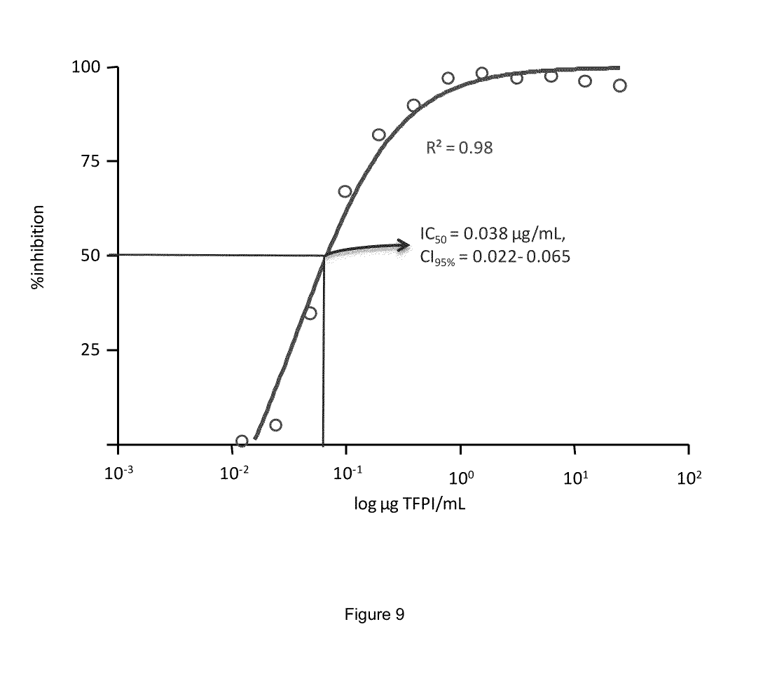

FIG. 9 shows a competition curve for the measurement of FXa in the presence of TFPI in solution.

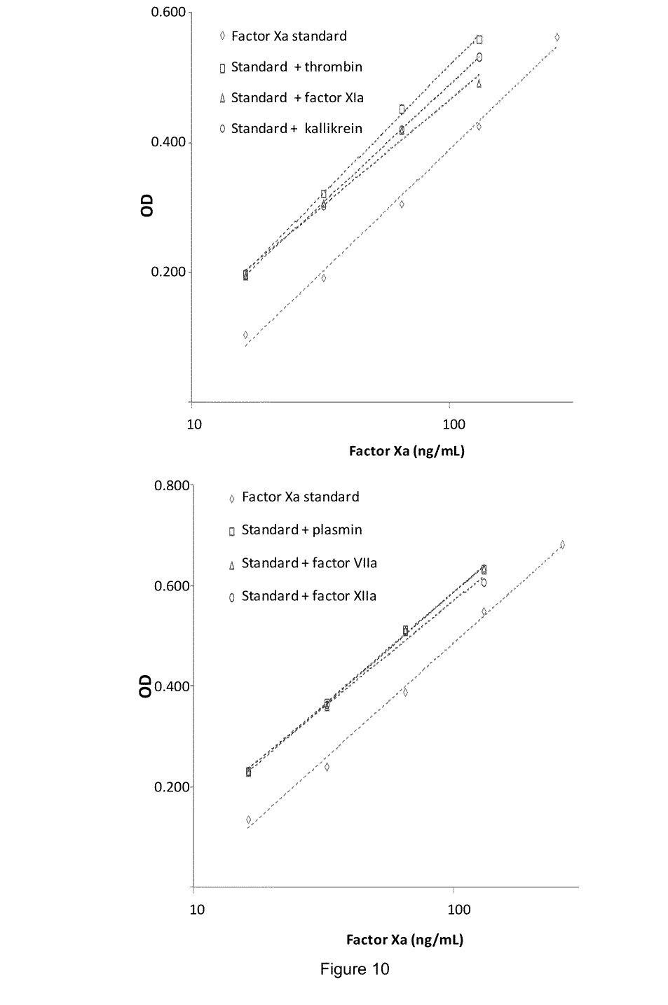

FIG. 10 shows dose-response curves for the measurement of FXa in the presence of other serine proteases.

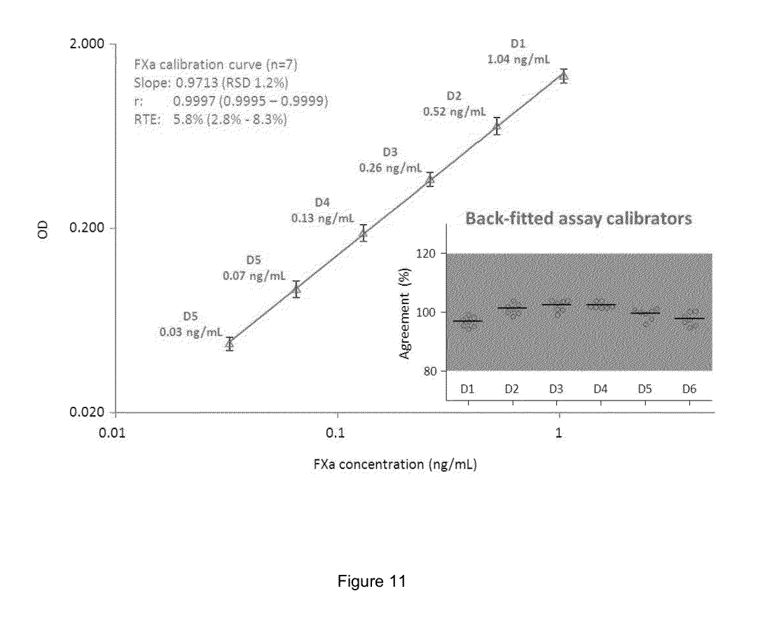

FIG. 11 gives the mean (n=7) sensitive calibration curve, suitable for the measurement of FXa concentrations in aPCCs.

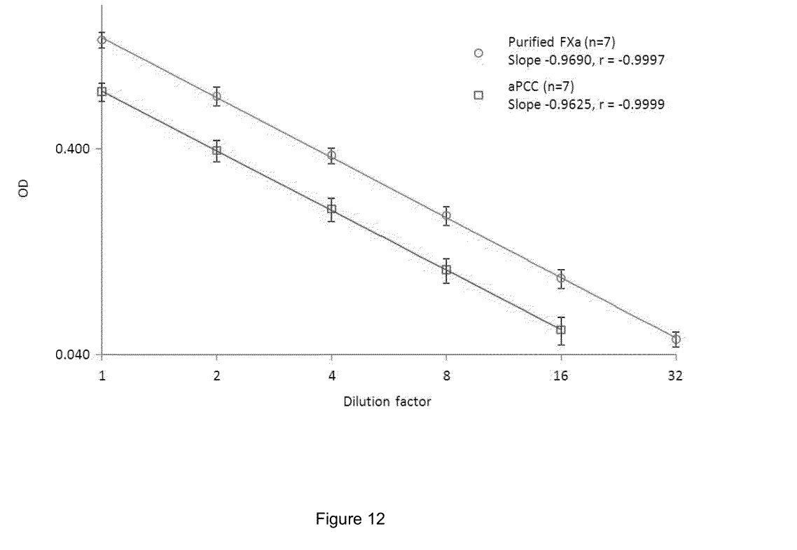

FIG. 12 shows the mean (n=7) dilution-response curves of purified FXa, used as assay calibrator, and the aPCC #1, as representative sample.

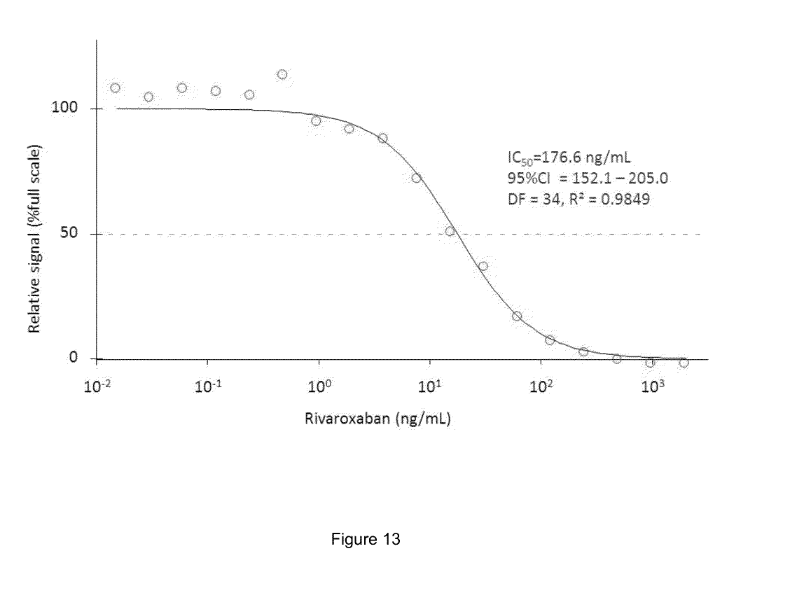

FIG. 13 shows an inhibition curve obtained for FXa in the presence of the direct FXa inhibitor Rivaroxaban.

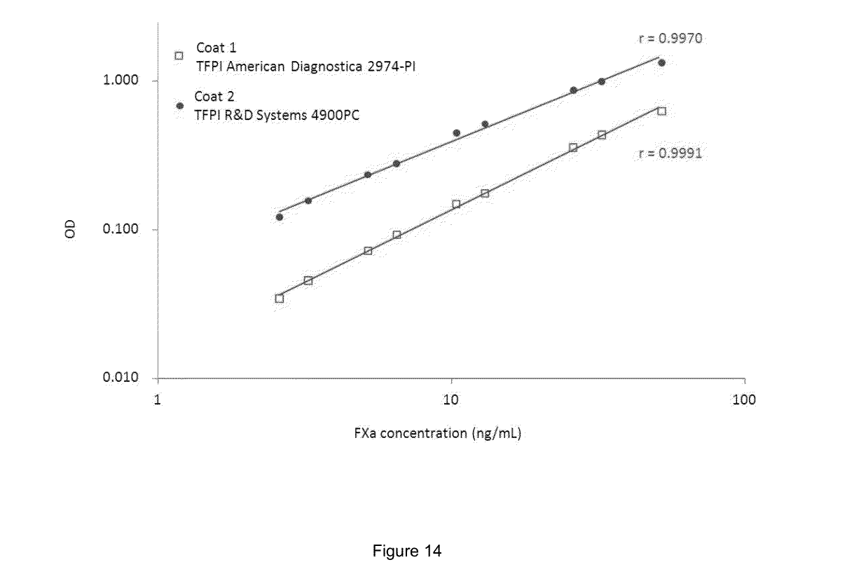

FIG. 14 shows the FXa calibration curves obtained for two brands of TFPI, ranging from 2.6 to 52 ng/mL.

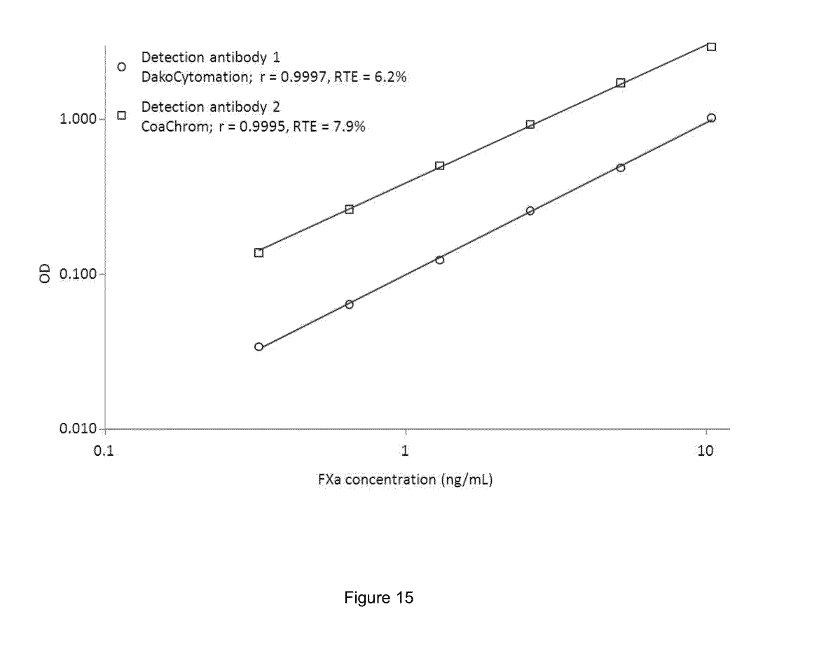

FIG. 15 shows the two FXa calibration curves obtained for two brands of FX detection antibody, ranging from 10.4 to 0.33 ng/mL.

The present invention will be further illustrated in the following examples without any limitation thereto.

### EXAMPLES

### Example 1

### Preparation and Characteristics of an Assay Calibration Curve for the Measurement of FXIIa Using Plate-Bound Corn Trypsin Inhibitor

Corn trypsin inhibitor (CTI) is a well-described, very selective inhibitor of FXIIa. Here, plate-bound CTI was used for the selective measurement of FXIIa. The assay calibration curve consisted of five serial 1+1 dilutions obtained by using a purified, commercially available FXIIa preparation (Factor alpha-XIIa, ERL #2590AL; 1 mg/mL). This calibration curve covered a range from 0.6 to 10 ng FXIIa/mL and was obtained as follows: Corn trypsin inhibitor (CTI from ERL, #334L; 1 mg/mL) was diluted to 20 μg/mL with phosphate-buffered saline (PBS; 8 g/L NaCl, 0.2 g/L KCl, 0.2 g/L KH₂PO₄, 1.26 g/L Na₂HPO₄×2H₂O, native pH) and incubated with the wells of a NUNC Maxisorp F96 plate at 0 to +10° C. overnight (100 μL/well). The plate was then washed with PBS containing 0.05% Polysorbate 20 (=washing buffer, WB) and inactivated by incubation with 200 μL/well washing buffer containing 10 mg/mL human serum albumin (Baxter) (=dilution buffer, DB) at 37° C. for 60 min. 100 μL/well dilution buffer was added to the wells and the pre-diluted FXIIa standard or FXIIa standard in 1/20-diluted IgG preparation Gammagard Liquid LE12E009 were loaded and serially diluted directly on the plate. The wells A1 to A12 contained only dilution buffer and served as blanks. The plate was then incubated at room temperature (RT, 18-26° C.) for 60 min and washed afterwards. Then, sheep anti-human FXII peroxidase (The Binding Site), diluted 1/1,000 in DB, was applied to the wells at 100 μl/well, incubated at RT for 60 min and removed by a washing step. The washed plate was then incubated with the ready-to-use trimethylbenzidine peroxidase reagent SureBlue (KPL; 100 μL/well) and incubated at RT until appropriate color development. The reaction was then stopped with 100 μL/well of 1.5 M sulfuric acid. Within 60 min, the plate was then measured in an ELISA reader at 450 nm with a reference measurement at 620 nm. The calibration curve was finally obtained by using a log-log fitting of the blank-corrected optical densities (ODs) measured and the FXIIa concentrations of the assay calibrators. FIG. 1 shows a 5-point calibration curve in buffer and in the presence of 2.5 mg/mL human IgG. The calibration curve characteristics slope, y intercept and correlation coefficient were very similar for the two curves irrespective of whether they were prepared in buffer or in IgG matrix. This was true also for the accuracy and the precision of the two curves.

The calibration curves met accepted requirements for accuracy, precision and linearity and were thus deemed to be appropriate for extrapolating samples. The range of linear relation between signal and FXIIa concentration went down to 0.6 ng/mL in the two matrices investigated. The back-fitted concentrations of this assay standard with the lowest FXIIa concentration made up 98.3% and 106.7% of the nominal one for the curves prepared in buffer and IgG matrix, respectively. This demonstrated that the high IgG concentration in the test sample did not obviously impact the assay performance. A similar conclusion could be drawn when the slopes of the two calibration curves were compared, which differed by less than 5%. These data demonstrate that a sensitive measurement of FXIIa was possible in samples containing high concentrations of human IgG.

### Example 2

### Measurement of FXIIa in the Presence of FXIa and Kallikrein

The selectivity of the measurement of FXIIa with the solid phase-bound inhibitor complex formation assay was shown using FXIa and kallikrein, two proteases of the contact activation system with similar activity profiles when using amidolytic substrates for their measurement. FIG. 2 shows the dose-response curves for the purified FXIIa preparation, mixtures of FXIIa with kallikrein and FXIa, as well as for samples containing only kallikrein and FXIa. The following purified preparations were used to obtain the test samples and mixtures: FXIIa (Factor alpha-XIIa, ERL #2590AL; 1 mg/mL), kallikrein (American Diagnostica 473, #001011, 1.2 mg/mL) and FXIa (American Diagnostica HFXIa 1771P, 0.51 mg/mL). The mixtures we prepared and measured containing a 3-fold and 2-fold excess of kallikrein and FXIa over FXIIa, respectively. FIG. 2 shows the dose-response curves obtained for these samples and gives their slopes when applicable as a measure to detect any impact of kallikrein and FXIa on the assay performance.

Samples of kallikrein and FXIa alone gave no response when measured at the same concentrations as FXIIa. Expectedly, this reflected the narrow inhibitory specificity of corn trypsin inhibitor which is known to selectively inhibit FXIIa while not affecting the activity of FXIa or kallikrein. The data shown here confirmed that the immobilization process resulting in plate-bound corn trypsin inhibitor did not change its specific inhibitory profile. This was further confirmed by the results obtained on measuring the mixtures: Only slight differences in slopes were found, less than 8%, which reflect assay variability rather than a pronounced telling difference between the curves obtained for the mixtures and FXIIa alone. When the FXIIa-alone curve was used for extrapolating the FXIIa concentrations of the mixtures, 101.2% and 104.5% of the nominal FXIIa concentrations were determined in the samples with 2- and 3-fold excess of FXIa and kallikrein, respectively. These data support the high selectivity of the assay format making an accurate and sensitive measurement of FXIIa possible in the presence of excess FXIa and kallikrein.

### Example 3

### Preparation and Characteristics of an Assay Calibration Curve for the Measurement of FXIa Using Plate-Bound C1-Inhibitor and Response for Kallikrein and FXIIa

C1-inhibitor (C1-inh) is a well-described inhibitor of FXIa, forming an irreversible stable C1-inhibitor-FXIa complex. Here plate-bound C1-inh was used for the selective measurement of FXIa. The assay calibration curve consisted of five serial 1+1 dilutions obtained by using a purified, commercially available FXIa preparation (FXIa #2581, ERL, 610 μg/mL). This calibration curve covered a range from 4.8 to 76 ng FXIa/mL and was obtained as follows: C1-inhibitor (Baxter, #79209108S; 50 U/mL) was diluted 1/100 with phosphate-buffered saline (see Example 1) and incubated with the wells of a NUNC Maxisorp F96 plate at 0 to +10° C. overnight (100 μL/well). The plate was then washed with PBS containing 0.05% Polysorbate 20 (=washing buffer, WB) and inactivated by incubation with 200 μL/well washing buffer containing 10 mg/mL human serum albumin (Baxter) (=dilution buffer, DB) at 37° C. for 60 min. 100 μL/well dilution buffer was added to the wells. Then, the purified FXIa standard, purified FXIIa (HFXIIA, #P2522P, Loxo, 1 mg/mL) and purified kallikrein (HPKa 1710AL, ERL, 1 mg/mL) were loaded and serially diluted directly on the plate. The wells only containing dilution buffer served as the assay blanks. The plate was then incubated at room temperature (RT) for 60 min and washed afterwards. Then, sheep anti-human FXI peroxidase (The Binding Site, CUS1613), diluted 1/1,000 in DB, was applied to the wells at 100 μl/well, incubated at RT for 60 min and removed afterwards by a washing step. The washed plate was then incubated with the ready-to-use trimethylbenzidine peroxidase reagent SureBlue (KPL; 100 μL/well) and incubated at RT until appropriate color development. The reaction was then stopped with 100 μL/well of 1.5 M sulfuric acid. Within 60 min, the plate was then measured in an ELISA reader at 450 nm with a reference measurement at 620 nm. The calibration curve was finally obtained by using a log-log fitting of the blank-corrected optical densities (ODs) measured and the FXIa concentrations of the assay calibrators. FIG. 3 shows the 5-point calibration curve obtained for the dilution series of FXIa and the response measured for the purified protease kallikrein and FXIIa.

An appropriate calibration curve was found ranging from 4.8 to 76 ng FXIa/mL with adequate accuracy as shown by the low relative total error RTE of 9.9%. RTE, a stringent measure for the quality of fit, was calculated according to RTE=(I x̅_{N}− x̅_{M}I+2SD)/ x̅_{M}×100. x̅_{N} and x̅_{M} represent the nominal and the measured mean, respectively, and SD the standard deviation of x̅_{M}. In order to obtain the mean x̅_{M}, the mean ODs of the individual dilutions were back-fitted on the curve, normalized by multiplication with the respective dilution and finally averaged. In contrast, neither kallikrein nor FXIIa elicited measurable signals in the concentrations investigated. This confirmed the selectivity of the approach applied for the measurement of FXIa.

### Example 4

### Measurement of FXIa Using Plate-Bound C1-Inhibitor in the Presence of Kallikrein and FXIIa

C1-inh is known to form irreversible complexes not only with FXIa but also with kallikrein and FXIIa. To address this feature of C1-inh which could possibly result in an unwanted assay interference caused by the competition for the plate-bound C1-inh, it was checked whether or not mixtures of FXIa with kallikrein and FXIa with FXIIa behaved similarly as FXIa alone. Thus, two FXIa mixtures were prepared containing a 2- and a 15-fold excess of FXIIa and kallikrein, respectively. FIG. 4 shows the dilution-response curves obtained and gives the agreement of the back-fitted assay standards as a measure for the accuracy.

The data obtained demonstrated that neither the presence of FXIIa nor the presence of kallikrein had any influence on the measurement of FXIa. This was shown by the slopes of the dose response curves determined for FXIa which differed by less than 2.5% in presence of kallikrein and FXIIa from that obtained for FXIa alone. Furthermore, the FXIa concentrations determined for the mixtures containing a 2- and 15-fold surplus of FXIIa and kallikrein were 104.4% and 103.3% of the corresponding nominal ones. This data confirmed the selectivity of the approach, which was in that case mainly determined by the detection antibody used for detecting the FXIa-C1-inhibitor complex formed on the solid phase.

### Example 5

### Calibration Curve for the Measurement of Kallikrein Using Plate-Bound C1-inhibitor

Purified C1-inh (Berinert, #19461711E, 50E/ml) was diluted 1/100 in PBS and 100 μL/well were coated as described before (Example 1). PBS containing 5 mg/mL gelatin (Sigma) was used to inactivate the plates and to dilute the samples and the detection antibody. A purified kallikrein preparation (American Diagnostica 473, #100316, 1.27 mg/mL) and the anti-human prekallikrein-peroxidase (TBS PP523.X, 1:1000 diluted) were used, while all other steps were done as described before (Example 1). FIG. 5 shows the calibration curve obtained. This curve ranged from 7.9 to 63.5 ng kallikrein/mL. Its correlation coefficient r and its RTE qualified the curve to be accurate and precise to be used for the measurement of kallikrein.

### Example 6

### Measurement of Activated Factor X (FXa) Using Plate-Bound Tissue Factor Pathway Inhibitor

Tissue factor pathway inhibitor (TFPI) is a potent high molecular weight inhibitor of FXa with several parts of the molecule involved in optimum inhibition. Data support that the complex formation between TFPI and FXa involves generating a 1:1 stoichiometric complex. The TFPI preparation from American Diagnostica (#4900PC) was used at a concentration of 1 μg/mL in PBS for coating the plates as given for Example 1. Purified human FXa supplied by HTI (#AA0203) was used with a concentration of 10.4 mg/mL as the FXa standard. The dilutions of the dilution series, ranging from 5,200 to 3.3 ng/mL, were prepared with PBS containing 5 mg/mL gelatin and 2 mM EDTA (=dilution buffer, DB). As a detection antibody, the rabbit anti-human factor X-peroxidase (DakoCytomation P0379), diluted 1/1,000 with DB was used. All further steps of the assay including the inactivation of the plates, the incubation of the wells with the samples and the detection antibody as well as the color development were done as described before (Example 1). FIG. 6 shows the dose-response relation by plotting the blank-corrected optical densities (ODs) versus the FXa concentrations of the dilution series. Also, it gives the data for the concentration range from 3.3 to 52 ng/mL, where a linear relation was obtained between the logarithms of FXa concentration and response.

Following a plateau, where because of the high FXa concentrations incubated with the plate a saturation of the plate-bound TFPI seemed to occur, a FXa concentration range (3.5 to 52 ng/mL) was identified, where there was a good linear relation between the logarithms of the FXa concentrations and the response measured. Thus, a correlation coefficient of r=0.9977 with an RTE of 15.3% was determined for this regression curve. All individual back-fitted concentrations of the five assay standards differed by not more than 12% from their respective nominal values. Thus, they complied with current guidelines used for evaluating the quality of fit of the calibration curves. Based on these data and using the conditions described above, a calibration curve based on the activity of the purified FXa preparation was set up. This preparation had a labeled activity of 11,648 U/mL and a 6 point calibration curve ranging from 0.36 to 11.6 mU/mL was defined. FIG. 7 shows four representative calibration curves, while the insert shows the agreement of the corresponding back-fitted concentrations of the calibration curve points with the nominal ones. Table 1 gives the original data and the calibration curve characteristics slope, y-intercept, correlation coefficient r and RTE determined for these curves.

TABLE 1
Calibration curves for the FXa-TFPI complex assay

| Test No. | D1 | D2 | D3 | D4 | D5 | D6 | Blank | Slope | Interc. | r | RTE |
|---|---|---|---|---|---|---|---|---|---|---|---|
| AE-1481 | 1.431 | 0.787 | 0.415 | 0.224 | 0.114 | 0.057 | 0.020 | 0.9308 | −0.821 | 0.9996 | 7.2 |
| AE-1482 | 1.517 | 0.832 | 0.429 | 0.229 | 0.111 | 0.061 | 0.030 | 0.9378 | −0.807 | 0.9997 | 6.5 |
| AE-1483 | 1.500 | 0.824 | 0.472 | 0.248 | 0.122 | 0.062 | 0.034 | 0.9209 | −0.783 | 0.9990 | 11.9 |
| AE-1484 | 1.394 | 0.742 | 0.399 | 0.207 | 0.105 | 0.071 | 0.045 | 0.8832 | −0.810 | 0.9978 | 17.4 |
| Mean | 1.461 | 0.796 | 0.429 | 0.227 | 0.113 | 0.063 | 0.032 | 0.9182 | −0.805 | 0.9990 | 10.8 |
| RSD | 4.0 | 5.2 | 7.3 | 7.4 | 6.3 | 9.4 | 32.1 | 2.6 | −2.0 | n.a. | n.a. |

Remarks:
n.a. stands for not applicable.

The calibration curve data qualified the FXa measurement with plate-bound TFPI as accurate and precise and as applicable to be used as a standard assay. Thus, the low variability of slope and intercept with RSDs of less than 3% indicated that the coating procedure provided comparable concentrations of active TFPI on the plates. The quality of fit of the calibration curves was also adequate in all cases, shown by the good correlation coefficients, the low RTEs and the back-fitted concentrations of the calibration curve standards, which differed by less than 20% from their nominal concentrations. Thus, the data confirmed that plate-bound TFPI can be used for the selective and sensitive measurement of mU-concentrations of FXa.

### Example 7

### Measurement of FXa Using Plate-Bound TFPI in the Presence of FX Zymogen

Next, it was checked whether or not the presence of FX zymogen would impact the FXa measurement with plate-bound TFPI. Therefore, an activated prothrombin complex concentrate (APCC) was analyzed, which is known to contain low concentrations of FXa but also high concentrations of the non-active FX zymogen. The assay was run essentially as described above. FIG. 8 shows the dose-response curves for three lots of APCC, measured at a starting dilution of 1/40 and gives their correlation coefficients and their slopes relative to that of the purified FXa preparation.

The slopes of the dose-response curves obtained for the three lots of APCC did not indicate any interference caused by the concentrations of FX zymogen present in the APCC preparation, which was in all cases higher than 10 U/mL. Thus, these curves had good linearity with r≧0.993 and their slopes differed by not more of 10% from that of the purified FXa preparation, used for the assay standardization. When extrapolating the responses on the calibration curve, FXa concentrations of 128 and 120 mU/mL were found for the APCC lots VNF2L027 and VNF2L030A. Reasonably and in line with the limitations of the chromogenic measurement, this was lower than the results obtained with a chromogenic FXa activity measurement, where 167 and 169 mU/mL were determined but still the data were in the same order of magnitude. Overall, the results confirmed that even a 100-fold surplus of the non-active FX zymogen did not interfere with the FXa measurement using plate-bound TFPI.

### Example 8

### Measurement of FXa in the Presence of TFPI in Solution

This example illustrates the specificity of the binding of FXa to plate-bound TFPI. In particular, mixtures of FXa (containing about 10 U/mL FXa) with gradually decreasing concentrations of TFPI ranging from 25 to 0.012 μg/mL were prepared and incubated at RT for 30 min. Then the assay was run as described above except that 5 mM EDTA was used in the dilution buffer instead of 2 mM. FIG. 9 shows the competition curve obtained for these samples.

The incubation of FXa with TFPI in solution resulted in FXa-TFPI complex formation thus reducing the concentration of FXa in solution. Consequently, complex formation with the plate-bound TFPI was also reduced and clearly reduced assay responses were obtained dependent on the TFPI concentration in solution. Thus, a typical competition curve with a clear plateau of competition reached by TFPI concentrations of higher than 1 μg/mL was obtained. 50% inhibition (IC₅₀) was observed under these conditions at a TFPI concentration of 0.038 μg/mL (95% confidence interval 0.022-0.065 μg TFPI per mL solution). These data confirmed the specificity of the approach for the measurement of FXa and furthermore showed that preformed FXa-TFPI complex, present in the test sample, had no influence on the assay performance.

### Example 9

### Influence of Plasmin and Aprotinin on the Measurement of FXa with Plate-Bound TFPI

There are several reports that plasmin causes proteolysis of circulating TFPI or cell-bound TFPI. Therefore, the effects of plasmin on the FXa measurement with plate-bound TFPI were checked and it was further investigated whether or not such effects, if observed, can be overcome by the addition of the plasmin inhibitor aprotinin. TFPI-coated plates were prepared as described above (Example 1). As a dilution, PBS containing 1 mg/mL bovine serum albumin (Sigma) and 5 mM EDTA, to which 150 KIU aprotinin/mL were added, was used here when its influence was checked. The purified plasmin preparation that was used for the spiking to FXa was HPlasmin 2026L (ERL), available at a concentration of 1 mg/mL. A FXa solution containing 0.52 μg/mL was spiked with 25 μg/mL plasmin and this sample was serially diluted with the two dilution buffers with and without 150 KIU/mL aprotinin. In addition, the plasmin solution used for spiking was also measured. Table 2 gives the blank-corrected ODs and the slopes and correlation coefficients of the dose-response curves when applicable.

TABLE 2
Influence of plasmin and aprotinin on the FXa measurement with
plate-bound TFPI

| FXa | Without aprotinin | | | With aprotinin (150 KIU/mL) | | |
|---|---|---|---|---|---|---|
| ng/mL | FXa | Plasmin | FXa + plasmin | FXa | Plasmin | FXa + plasmin |
| 520.0 | 0.791 | 0.000 | 0.010 | 0.894 | 0.000 | 1.069 |
| 260.0 | 0.653 | 0.001 | 0.013 | 0.752 | 0.000 | 0.886 |
| 130.0 | 0.468 | 0.000 | 0.017 | 0.560 | 0.000 | 0.631 |
| 65.0 | 0.314 | 0.000 | 0.032 | 0.359 | 0.000 | 0.423 |
| 32.5 | 0.179 | 0.000 | 0.040 | 0.200 | 0.000 | 0.234 |
| Slope | 0.5192 | n.a. | n.a. | 0.5915 | n.a. | 0.708 |
| % slope | 100.0 | n.a. | n.a. | 113.9 | n.a. | 119.8 |
| r | 0.9987 | n.a. | n.a. | 0.9985 | n.a. | 0.9986 |

The data obtained were as expected: The purified plasmin preparation did not show any signal in both approaches, diluted without and with aprotinin but clearly reduced the signal measured for the FXa preparation when added to the FXa preparation. This was not the case when aprotinin was included in the dilution buffer. The addition of aprotinin to the dilution buffer protected plate-bound TFPI from inactivation by plasmin but on the other hand did not impede the complex formation between FXa and TFPI. In contrast, the slightly higher ODs measured for the FXa preparation in presence of aprotinin favor the assay by increasing its sensitivity. Thus, the addition of aprotinin (150 KIU/mL) not only prevented the detrimental interaction of TFPI with plasmin but also increased the assay sensitivity by about 10% as far as the direct readouts were concerned.

### Example 10

### Measurement of FXa in the Presence of the Proteases Plasmin, FVIIa, FXIIa, Thrombin, FXIa and Kallikrein

After having established the beneficial effects of aprotinin on the assay performance, the assays performance was further investigated in the presence of other proteases. In particular, the influence of the plasma serine proteases plasmin, factor Vila, factor XIIa, thrombin, factor XIa and kallikrein was checked. TFPI-coated plates were prepared and inactivated as described (Example 1), using as dilution buffer PBS containing 5 mg/mL gelatin, 2 mM EDTA and 100 KIU/mL aprotinin. Furthermore, the following purified, commercially available proteases were used: Human plasmin, ERL 2026L, 1 mg/ml; Novo Seven, E457808, 1.2 mg/ml; human FXIIa, ERL1660AL, 1 mg/ml; Thrombin 5591R00A, 39 IU/ml; human FXIa, ERL 1771P, 0.51 mg/ml; and human kallikrein, ERL 2680AL, 1 mg/ml. FXa solution (0.52 μg/mL) was mixed with the proteases so that the non-FXa protease was present in the reaction mix in a 5- to 10-fold excess over FXa. In addition, the non-FXa proteases were measured at concentrations ranging from 2.6 up to 6 μg/mL.

FIG. 10 gives the dose-response curves obtained for the mixtures of FXa with other serin proteases, while Table 3 gives the slopes of the dose-response curves, their slopes relative to those of the FXa standards, the FXa concentrations measured for the respective mixtures and the recovery of FXa as a percent of the nominal FXa concentration. As expected, none of the six serine proteases, measured at concentrations of as high as 6 μg/mL gave any signal which could be clearly discriminated from the blank signal. This furthermore demonstrated the excellent selectivity of the FXa measurement using plate-bound TFPI. This high selectivity is reached by the specific complex formation used to capture FXa from complex mixtures and further enhanced by the specific detection of the FXa molecule in complex with TFPI.

TABLE 3
Relative slopes and FXa concentrations measured in presence of other
serine proteases

| Parameter | FXa Std1 | +Plasmin | +FVIIa | +FXIIa | FXa Std2 | +Thrombin | +FXIa | +Kallikrein |
|---|---|---|---|---|---|---|---|---|
| Slope | 0.4664 | 0.4491 | 0.4544 | 0.4245 | 0.3822 | 0.3709 | 0.3322 | 0.3701 |
| % slope | 100.0 | 96.3 | 97.4 | 91.0 | 100.0 | 97.0 | 86.9 | 96.8 |
| r | 0.9978 | 0.9993 | 0.9994 | 0.9971 | 0.9969 | 0.9957 | 0.9956 | 0.9997 |
| FXa | 10.4 | 9.0 | 8.9 | 8.9 | 10.4 | 10.9 | 9.8 | 9.7 |
| (mg/mL) | | | | | | | | |
| % recovery | 100.0 | 86.5 | 85.6 | 85.6 | 100.0 | 104.8 | 94.2 | 93.3 |

The quantitative data confirmed that all non-FXa serine proteases investigated had no influence on the measurement of FXa using the plate-bound TFPI complex formation. Thus, the slopes of the dose-response curves determined for the mixtures of FXa with the other serin proteases differed by less than 15% from those obtained for the respective FXa standards. The highest difference of almost 15% was found for the mixture containing FXIa, whereas all the slopes of all other mixtures differed by less than 10%. The linearity of these dose-response curves was acceptable with all correlation coefficients r higher than 0.995. The recovery of FXa in the mixtures was also acceptable with recoveries of better than 85% in all cases. This is acceptable considering the high dilution factors required to measure the high FXa concentration of the sample used for the preparation of the mixture. All in all, the data demonstrated that even high concentrations of non-FXa serine proteases did not interfere with the assay.

The following Examples 11 to 15 focus on the selective measurement of FXa in the complex matrix of an activated prothrombin complex concentrate.

### Example 11

### Sensitive and Selective Measurement of Activated Factor X (FXa) in the Matrix of an Activated Prothrombin Complex Concentrate (aPCC)

The sensitivity of the FXa assay described in the Examples 6 to 10 was increased to be suitable for the measurement of FXa in the complex protein matrix of an activated prothrombin complex concentrate. TFPI (R&D Systems, Nr.2974-PI) was coated to Maxisorp F96 plates at a concentration of about 1 μg/mL in PBS overnight. The washed plates were then inactivated with dilution buffer (PBS containing 0.05% Polysorbate 20, 5 mg/mL non-fat dry milk, 3 mM EDTA, 120 KIE Aprotinin) by incubating the plates at 37° C. for 60 min, washed again and incubated with the dilution series of the purified FXa preparation used as an assay standard and the aPCC samples. The dilution series consisting of six serial 1+1 dilutions were prepared using dilution buffer: The starting dilution of these dilution series were 1×10⁷ and 1/200 for the purified FXa preparation from HTI (#AA0203; FXa concentration 10.4 mg/mL) and the aPCC preparations, respectively. The resulting FXa calibration curve covered a FXa concentration range from 0.03 to 1.04 ng/mL and was sensitive enough to reproducibly allow measuring the FXa concentrations in aPCC. The dilutions were then incubated at RT for 60 min, before the anti-FX detection antibody (rabbit-anti human FX peroxidase; CoaChrom RAFX HRP; 1/250) was added to the washed plate and again incubated at RT for 60 min. The further processing of the plate, and in particular the color reaction, is described in Example 1.

FIG. 11 gives the mean (n=7) sensitive calibration curve, suitable for the measurement of FXa concentrations in aPCCs. The six-point calibration curve covered a FXa concentration range from 0.03 to 1.04 ng/mL and could be reproducibly constructed, as evidenced by the low inter-day RSD of 1.2% determined for the mean slope of the calibration curves. The relative total error (RTE) of the curves was calculated according to RTE=(|x_{F}−x_{N}|+2SD)/x_{N}×100. x_{F} and x_{N} stand for the mean of the back-fitted, dilution-corrected concentrations of the calibration curve standards and its nominal concentration, respectively. SD gives the standard deviation of x_{F}. The mean RTE was only 5.8% with individual values ranging from 2.8% to 8.3%. Also, the back-fitted assay calibrators agreed well with their nominal concentrations with all individual values within a ±6% range. These data demonstrated that the sensitive FXa calibration curve could be reproducibly established with very similar quality attributes as far as the calibration curve fitting was concerned.

Table 4 shows the FXa concentrations in ng/mL, measured for five lots of an aPCC in duplicates within one run. The aPCC lots #3 and #5 were formulated to contain half of the potency of the three other lots.

TABLE 4
Intra-assay precision of the FXa measurement in an aPCC

| aPCC | [1] | [2] | Mean | Δ% |
|---|---|---|---|---|
| #1 | 110.8 | 111.1 | 111.0 | 0.3 |
| #2 | 108.2 | 107 | 107.6 | 1.1 |
| #3 | 41.9 | 44.2 | 43.1 | 5.3 |
| #4 | 137.9 | 132.9 | 135.4 | 3.7 |
| #5 | 42.3 | 44.0 | 43.2 | 3.9 |

Remarks:
The FXa concentrations are given in ng/mL.

The relative difference between the duplicate measurements ranged from 0.3% to 5.3%. This was within the magnitude usually observed for ligand-binding assays and qualified the assay to provide precise data within a run. Table 5 shows the results of repeated measurements, done in six independent runs. Again, the FXa concentrations are given in ng/mL. The aPCC lots #3 and #5 were formulated to contain half of the potency of the three other lots.

TABLE 5
Inter-assay precision for the FXa measurement in an aPCC

| | Run | Run | | | | | | |
|---|---|---|---|---|---|---|---|---|
| aPCC | 1 | 2 | Run 3 | Run 4 | Run 5 | Run 6 | Mean | RSD |
| #1 | 119.1 | 112.1 | 119.5 | 110.8 | 112.9 | 107.0 | 113.6 | 4.3 |
| #2 | 109.7 | 112.4 | 114.1 | 108.2 | 105.9 | 103.4 | 109.0 | 3.7 |
| #3 | 45.3 | 45.9 | 46.8 | 41.9 | 43.2 | 42.0 | 44.2 | 4.7 |
| #4 | 101.0 | 112.4 | 104.1 | 137.9 | 125.3 | 122.4 | 117.2 | 11.9 |
| #5 | 46.1 | 46.5 | 47.7 | 42.3 | 45.7 | 41.7 | 45.0 | 5.4 |

Remarks:
The FXa concentrations are given in ng/mL.

Four of the five samples showed an inter-assay precision of less than 5.5%, expressed as the RSD of the mean of six independent measurements. Only one sample had an RSD of higher than 10%. Including this aberrantly performing sample for calculating the mean RSD resulted in an average RSD of 6.0%. This low RSD provided evidence that the FXa measurement could be done at a precision required to measure FXa in an aPCC. FIG. 12 shows the mean (n=7) dilution-response curves of purified FXa, used as the assay calibrator, and the aPCC #1, as a representative sample.

The two dilution-response curves were very linear and highly parallel. Thus, the slope of the mean aPCC dilution-response curve differed by less than 1% from that of the purified FXa preparation. This demonstrated that not only high levels of FX zymogen as present in the aPCC but also the presence of other vitamin K-dependent coagulation factors had no detrimental influence on the sensitive and specific measurement of FXa in an aPCC.

### Example 12

### Influence of the Direct FXa Inhibitor Rivaroxaban on the FXa Measurement in a Purified System

The FXa measurement was basically done as described in Example 11. Samples containing 2 μg/mL purified FXa (HTI) were mixed with the direct FXa inhibitor Rivaroxaban (Santa Cruz sc-208311, dissolved in dimethyl sulfoxide) to obtain final concentrations of 20,000 to 0.15 ng/mL and incubated at RT for 30 min, before they were diluted 1/1000 and loaded to the TFPI-coated, inactivated plates. FXa samples containing buffer instead of Rivaroxaban were treated in the same way and served to define the response of FXa without inhibitor. FIG. 13 shows the inhibition curve obtained.

Pre-incubation of the purified FXa sample with the direct FXa inhibitor Rivaroxaban before running the FXa measurement resulted in a clear concentration-dependent reduction of the signal with signal extinction of higher than 95% caused by Rivaroxaban concentrations of higher than 1.25 μg/mL. Fitting of data points (degree of freedom, DF=34) resulted in an IC₅₀ of 176.6 ng/mL under the experimental setting used here. These data demonstrated the selectivity of the FXa measurement because the assay response could be inhibited dose-dependently by the specific, direct FXa inhibitor Rivaroxaban.

### Example 13

### Comparison of Two Brands of TFPI for Running the FXa Measurement

TFPI can reasonably be assessed to be a critical reagent for the assay. Therefore, two brands of TFPI were compared head-to-head with regard to their suitability for the measurement of FXa: TFPI obtained from American Diagnostica (4900PC) and obtained from R&D Systems (2974-PI). The two preparations were used at the same concentration of 1 μg/mL for coating the plates. For this particular comparison, the dilution buffer used was PBS containing 0.05% Polysorbate 20, 5 mg/mL gelatin, 2 mM EDTA and 100 KIE aprotinin, while the assay itself was carried out as described in Example 11. FIG. 14 shows the FXa calibration curves obtained, ranging from 2.6 to 52 ng/mL.

Using the two TFPI preparations, coated at the same protein concentration based on the information provided by the manufacturer, generated calibration curves of similar linearity as shown by their correlation coefficients. Also, the blanks only differed marginally with 0.012±0.002 (n=12) and 0.016±0.004 (n=12) for the TFPI preparations obtained from American Diagnostica and R&D Systems, respectively. The signal intensities, however, were clearly higher for the TFPI preparation provided by R&D Systems, while the ratio between the blank and the highest signal was lower (1.2% for the R&D Systems TFPI versus 2.0% for the American Diagnostica). The data, however, showed that both preparations can be used for the FXa measurement because the slight differences in signal can be compensated by adjusting the concentration of the detection antibody.

### Example 14

### Comparison of Two Brands of FX Detection Antibody

Apart from TFPI, specifically capturing FXa to the solid support, also the detection antibody, an anti-FX antibody, can be assessed to be a critical reagent as it has to bind to the plate-immobilized FXa-TFPI complex. Therefore, the applicability of two brands of polyclonal rabbit anti-human FX antibodies for this purpose was checked. The assay was carried out as described in Example 13 with regard to the dilution buffer. In particular, rabbit anti-human factor X/HRP from DakoCytomation (P0379; 1.3 mg/mL, diluted 1/1,000) and rabbit anti-human factor X/POD from CoaChrom (RAF X-HRP, 2 mg/mL; 1/1,500) were compared head-to-head. FIG. 15 shows the two FXa calibration curves obtained, ranging from 10.4 to 0.33 ng/mL.

Using the two anti-FX peroxidase preparations resulted in similar calibration curves as far as their linearity was concerned. This statement was not only based on the good correlation coefficients, but also on the low relative total errors (RTES), being a severe combined measure for accuracy and precision of a calibration curve. Their signal intensities, however, were clearly different with higher blank-corrected optical densities for the anti-FX peroxidase conjugate provided by CoaChrom. Nevertheless, the data showed that both preparations can be used for the FXa measurement because the differences in signal can be compensated by adjusting the incubation time for the peroxidase activity measurement or by increasing the concentration of the detection antibody.

### Example 15

### Measurement of FXa in the Presence of Other Serine Proteases

The experimental setting of the FXa assay was as given in Example 11. The following purified plasma serine proteases were added at a 10,000 fold excess in mass to purified human FXa (HTI): Plasmin (ERL #2026L; 1 mg/mL), recombinant factor Vila (FVIIa; Baxter, PIrFVIIa; 500 μg/mL), factor XIIa (FXIIa; ERL #1660AL; 1 mg/mL), Thrombin (FIIa; Baxter, #5591R00A; 39 U/mL), factor Xla (FXIa; ERL #1611; 0.59 mL) and kallikrein (KK; ERL #2680AL, 1 mg/mL). The FXa-serine protease mixtures were then loaded to the TFPI-coated, inactivated plate at FXa concentrations to obtain FXa dilution series ranging from 0.52 to 0.03 ng/mL. Table 6 shows the optical densities and the resulting regression curve parameters slope, y-intercept and correlation coefficient r for the dilution series of FXa and the FXa-serine protease mixtures. In addition, the slopes calculated for the FXa-protease mixtures are related to that of the FXa sample.

TABLE 6
Optical densities and calibration curve parameters of the FXa dilution
series containing other serine proteases

| | FXa | | | | | | |
|---|---|---|---|---|---|---|---|
| ng/mL | alone | +Plasmin | +FVIIa | +FXIIa | +FIIa | +FIXa | +KK |
| 0.52 | 1.159 | 1.175 | 1.166 | 1.169 | 1.207 | 0.978 | 1.288 |
| 0.26 | 0.595 | 0.592 | 0.582 | 0.575 | 0.621 | 0.518 | 0.645 |
| 0.13 | 0.304 | 0.298 | 0.302 | 0.290 | 0.308 | 0.279 | 0.327 |
| 0.07 | 0.142 | 0.140 | 0.141 | 0.144 | 0.155 | 0.151 | 0.166 |
| 0.03 | 0.069 | 0.066 | 0.068 | 0.069 | 0.069 | 0.077 | 0.084 |
| Slope | 1.0207 | 1.0409 | 1.0264 | 1.0165 | 1.0257 | 0.9111 | 0.9856 |
| y-intercept | 0.367 | 0.378 | 0.367 | 0.358 | 0.387 | 0.250 | 0.388 |
| r | 0.9996 | 0.9997 | 0.9997 | 0.9999 | 0.9993 | 0.9999 | 1.0000 |
| % slope | 100.0 | 102.0 | 100.6 | 99.6 | 100.5 | 89.3 | 96.6 |

The presence of high excesses of other serine proteases had no influence on the FXa measurement because the slopes of the dilution-response curves, calculated for the FXa-serine protease mixtures, differed by less than 11% from that of the sole FXa sample. In particular, only the FXIa-FXa mixture showed a slope differing by 10.7%, while all other mixtures differed by less than 4.5%. These data demonstrated the high selectivity of the TFPI-based FXa measurement because even the 10,000 fold excess of other plasma serine proteases did not result in detectable assay interference.

1. A method for the selective determination of the concentration of an active human protease coagulation factor in a sample, comprising the steps of:
(a) binding a specific inhibitor of the human protease coagulation factor to a solid phase;
(b) incubating the solid phase with the sample;
(c) incubating the solid phase with a specific detection reagent binding to the human protease coagulation factor;
(d) determining the amount of detection reagent bound to the solid phase; and
(e) determining the amount of active human protease coagulation factor in the sample from the amount of detection reagent determined in step (d).

2. The method of claim 1, further comprising the step of blocking the solid phase prior to step (b).

3. The method of claim 1, further comprising the step of diluting the sample prior to incubation with the solid phase in step (b).

4. The method of claim 1, further comprising the step of washing the solid phase prior to and/or after step (c).

5. The method of claim 1, wherein the human protease coagulation factor is selected from the group consisting of factor Xa (FXa), factor XIIa (FXIIa), factor XIa (FXIa), kallikrein, factor IIa (FIIa) and factor VIIa (FVIIa).

6. The method of claim 1, wherein the human protease coagulation factor and the corresponding specific protease inhibitor are selected from the group consisting of FXa and tissue factor pathway inhibitor (TFPI), FXIIa and corn trypsin inhibitor (CTI), FXIa and C1-inhibitor, kallikrein and C1-inhibitor, FIIa and heparin cofactor II, FVIIa and antithrombin.

7. The method of claim 1, wherein the solid phase is selected from the group consisting of microplates, gels, and microparticles.

8. The method of claim 1, wherein the specific detection reagent is an antibody.

9. The method of claim 8, wherein the antibody is conjugated to peroxidase.
